(19) United States
(12) Patent Application Publication
(10) Pub. No.: US 2009/0062691 A1
(43) Pub. Date: Mar. 5, 2009

(54) Specimen Collecting

(75) Inventor: Nicholas H. Kim, New York, NY (US)

Correspondence Address: FISH & RICHARDSON P.C., P.O. BOX 1022, MINNEAPOLIS, MN, 55440-1022, US

(73) Assignee: Quaternion Investments LLC, New York, NY (US)

(21) Appl. No.: 11/847,189

(22) Filed: Aug. 29, 2007

(51) Int. Cl. A61B 10/00

(52) U.S. Cl. 600/569; 600/573

(57) ABSTRACT

A device includes a tubular member sized for placement in the vaginal canal. The tubular member has a wall defining a longitudinal through-channel having a central axis, and an opening at a distal end oriented at an oblique angle to the central axis such that a first section of the wall extends distally of a distal end of a second section of the wall. The first section flares outwardly by 13-30 mm to an enlarged outer diameter at the opening. The oblique angle is 20-65°, and the first section of the wall extends distally of the distal end of the second section of the wall by 20-60 mm.

### BACKGROUND

This invention relates to specimen collecting.

The Pap test (a/k/a Pap smear, cervical smear, Papanicolaou smear) is the global standard of care for the early detection of cervical cancer. The Pap test is not intended to be a definitive diagnostic test, but rather a risk assessment-oriented, basic screening procedure. The Pap test is an anatomic pathology process, where human tissue, cells, and/or secretions from the site of a potential cancer—in this instance, the cervicovaginal region—are viewed under a microscope by a trained laboratory professional in search of morphologic (i.e., cell form/structure) changes that evidence—to varying degrees along a standardized continuum of severity (e.g., the Bethesda System)—the likely existence and progression of cervical cancer.

A “positive” Pap test—one where “suspicious” cellular changes have been identified—is generally followed-up with a colposcopy and/or definitive biopsy. Treatment options at the pre-cancerous stage are relatively simple and painless (minor sedation/discomfort), and generally one-time, cost-effective procedures (e.g., cryo- or laser-ablation of abnormal cells/lesions) that are performed on an outpatient/in-office basis and enjoy an extremely high, proven success rate in totally eradicating, and otherwise completely halting, the pre-cancerous growth and/or progression.

Importantly, given the inherently and otherwise unavoidably imprecise/imperfect nature of the Pap test, the Pap test owes its legendary efficacy in preventing cervical cancer as much, if not more, to the sheer repetitiveness of the procedure over the course of a woman's life, than to the sensitivity, per se, of any single Pap test. According to cytology experts, the statistical confidence level of an accurate Pap test result rises from a low of 60-70% to upwards of 98% after just three consecutive annual Pap tests.

For this reason, health care standards-setting organizations, such as the American Cancer Society, generally recommend regular (e.g., annual) Pap testing for all women, and for sexually active teens. However, according to a recent Gallup poll conducted by the College of American Pathologists (CAP), nearly 40% of those women polled had not had a Pap test within the past year.

### SUMMARY

It is believed that a barrier to having an annual Pap test is the physician, in-office, Pap test specimen collection procedure. The specimen collector of the present invention enables a woman to reliably collect her own Pap test specimen, wherever and whenever she chooses, of at least comparable quality to that which a trained physician can obtain in-office, based on the Bethesda System or equivalent specimen adequacy criteria. Once collected, the woman can prepare and transport/deliver her viable specimen to the medical establishment (either a physician or directly to a pre-determined CLIA— or other certified cytology lab) for professional processing and interpretation, just as if the specimen had been taken by her physician in-office.

The specimen collector is a simple, one-size-fits-all design for high volume, low cost manufacturing; and yet is robust enough to accommodate the range of anatomic variations in the female population. The specimen collector is attractive, comfortable and simple to use.

According to one aspect, the specimen collector includes a tubular member sized for placement in the vaginal canal. The tubular member has a wall defining a longitudinal through channel having a central axis. The tubular member defines an opening at a distal end oriented at an oblique angle to the central axis with a first section of the wall extending distally of a distal end of a second section of the wall. At least a portion of the first section of the wall flares distally to an enlarged outer diameter. The first section of the wall terminates at the opening, and the opening is bounded by a rounded edge.

Embodiments of this aspect may include one or more of the following features.

A circumference of the tubular member in at least a region of the tubular member flaring in opposite directions is asymmetric about the central axis. The asymmetric region has a change in shape along a length of the tubular member. The tubular member has a length in the range of 120 to 220 mm, and an outer diameter that flares from a smaller diameter in the range of 15-20 mm to a larger diameter in the range of 30-50 mm.

The specimen collector includes a sampling member configured for receipt within the tubular member that has a distal end comprising a specimen removing and retaining element. In an illustrated embodiment, the specimen removing and retaining element is a brush oriented off-axis relative to a longitudinal axis of the sampling member. One or both of the tubular member and the sampling member includes an orientation indicator.

According to another aspect, a specimen collector includes a tubular member sized for placement in the vaginal canal. The tubular member has a wall defining a longitudinal through channel terminating in a distal opening. The wall has an outer surface that is continuous to the distal opening and flares distally to a fixed, enlarged outer diameter. The longitudinal through channel has a central axis and a circumference of the tubular member in at least a region of the tubular member flaring in opposite directions is asymmetric about the central axis.

Embodiments of this aspect may include one or more of the following features.

The asymmetric region has a change in shape along a length of the tubular member. The distal opening is oriented at an oblique angle to the central axis. The specimen collector includes a sampling member configured for receipt within the tubular member.

According to another aspect, a method of collecting a specimen includes accessing the cervix by introducing a tubular member into the vaginal canal and collecting the specimen. The tubular member defines a longitudinal through channel having a central axis and an opening at a distal end oriented at an oblique angle to the central axis such that the opening faces the cervix and with a first section of the wall extending distally of a distal end of a second section of the wall. At least a portion of the first section of the wall flares distally to an enlarged outer diameter. The first section of the wall terminates at the opening.

Embodiments of this aspect may include one or more of the following features. The tubular member isolates the cervix from surrounding vaginal tissue. Collecting the specimen includes contacting the cervix with a sampling member received within the tubular member. For example, specimen is collected using a repetitive back and forth axial motion of the sampling member. The method includes dipping the sampling member in a collection medium. The tubular member is introduced into the vaginal canal without folding the tubular member. The specimen collected is cervical and vaginal cells and secretions.

According to another aspect, a device includes a tubular member sized for placement in the vaginal canal. The tubular member has a wall defining a longitudinal through channel having a central axis, and an opening at a distal end oriented at an oblique angle to the central axis such that a first section of the wall extends distally of a distal end of a second section of the wall. The first section flares outwardly by 13-30 mm to an enlarged outer diameter at the opening.

Embodiments of this aspect may include one or more of the following features. The oblique angle is 20-65°. The first section of the wall extends distally of the distal end of the second section of the wall by 20-60 mm.

According to another aspect, a device includes a tubular member sized for placement in the vaginal canal. The tubular member has a wall defining a longitudinal through channel having a central axis, and an opening at a distal end oriented at an oblique angle to the central axis such that a first section of the wall extends distally of a distal end of a second section of the wall to the opening by 20-60 mm without the first section converging toward the second section. The specimen collector further includes a sampling member for receipt within the tubular member and actuatable from outside the vaginal canal to contact the cervix.

Embodiments of this aspect may include one or more of the following features. The oblique angle is 20-65°. The first section flares outwardly by 13-30 mm.

According to another aspect, a method of collecting a specimen includes accessing the cervix by introducing a tubular member into the vaginal canal. The tubular member has a wall defining a longitudinal through channel having a central axis, and an opening at a distal end oriented at an oblique angle to the central axis such that a first section of the wall extends distally of a distal end of a second section of the wall to the opening by 20-60 mm without the first section converging toward the second section. The method includes collecting the specimen.

According to another aspect, a kit includes a tubular member sized for placement in the vaginal canal, a sampling member configured for receipt within the tubular member, and a container of collection medium suitable for preserving and transporting human tissue cells and related tissue secretions. The tubular member has a wall defining a longitudinal through channel having a central axis. The tubular member defines an opening at a distal end oriented at an oblique angle to the central axis with a first section of the wall extending distally of a distal end of a second section of the wall. At least a portion of the first section of the wall flares distally to an enlarged outer diameter at the opening.

Embodiments of this aspect may include one or more of the following features. The collection medium is PreservCyt®, SurePath® Perservative Solution, CytoRich®, or Rapid Capture® System STM. The tubular member has a length, for example, in the range of 120 to 220 mm, that is sufficient to extend from the cervix out of the vagina. The tubular member is non-foldable in use. A circumference of the tubular member in at least a region of the tubular member flaring in opposite directions is asymmetric about the central axis. The asymmetric region has a change in shape along a length of the tubular member. In a preferred configuration, the tubular member has an outer diameter that flares from a smaller diameter in the range of 15-20 mm to a larger diameter in the range of 30-50 mm.

The sampling member has a distal end comprising a specimen removing and retaining element, e.g., a brush oriented off-axis relative to a longitudinal axis of the sampling member. One or both of the tubular member and the sampling member includes an orientation indicator.

According to another aspect, a kit includes a tubular member sized for placement in the vaginal canal, a sampling member configured for receipt within the tubular member, and a container of collection medium. The tubular member has a wall defining a longitudinal through channel terminating in a distal opening. The wall has an outer surface that is continuous to the distal opening, and the wall flares distally to a fixed, enlarged outer diameter. The longitudinal through channel has a central axis and a circumference of the tubular member in at least a region of the tubular member flaring in opposite directions is asymmetric about the central axis.

Embodiments of this aspect may include one or more of the following features.

The asymmetric region has a change in shape along a length of the tubular member. The opening is oriented at an oblique angle to the central axis. The collection medium is PreservCyt®, SurePath® Perservative Solution, CytoRich®, or Rapid Capture® System STM. The tubular member has a length sufficient to extend from the cervix out of the vagina. The tubular member is non-foldable in use. The sampling member has a brush oriented off-axis. One or both of the tubular member and the sampling member includes an orientation indicator.

Advantageously, the shape and dimensions of the distal, insertion end of the tubular member provides for ease of insertion through the constraining introitus/opening of the vaginal canal without requiring folding of the tubular member and while still providing the desired stretching of the vaginal canal in the vicinity of the cervix.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an illustration of a specimen collection device in use.

FIG. 2 is an isometric view of the specimen collection device.

FIGS. 3-7 are side, top, bottom, and views of the specimen collection device.

FIG. 8 is a cross-sectional side view of a tubular member of the specimen collection device.

FIG. 9 is a cross-sectional view of the tubular member taken along lines 9-9 in FIG. 8 and shown without a handle of the tubular member for clarity.

FIG. 10 is a cross-sectional view of the tubular member taken along lines 10-10 in FIG. 8 and shown without the handle of the tubular member for clarity.

FIG. 11 is a cross-sectional view of the tubular member taken along lines 11-11 in FIG. 8 and shown without the handle of the tubular member for clarity.

FIG. 12 is an isometric view of a sampling member of the specimen collection device.

FIG. 13A illustrates the anatomy of the vaginal canal and cervix.

FIG. 13B illustrates the anatomy of the vaginal canal and cervix with a conventional, expanded speculum in place.

FIG. 13C illustrates the anatomy of the vaginal canal and cervix with the tubular member of FIG. 8 in place.

FIG. 14 is a view of the distal region of the specimen collection device in use.

FIG. 15 illustrates the sampling member being dipped in a collection medium.

FIG. 16 illustrates the specimen collection device as a component of a kit.

FIGS. 17a-17c illustrate an alternative embodiment of the tubular member.

FIGS. 18a-18c illustrate an another alternative embodiment of the tubular member.

### DETAILED DESCRIPTION

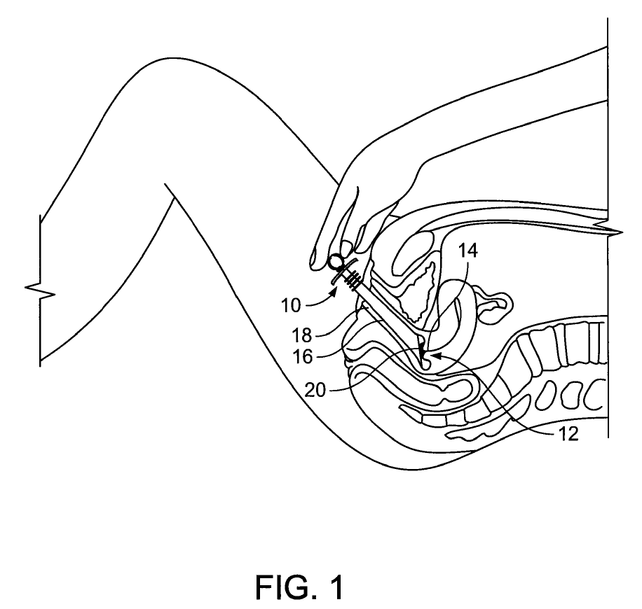

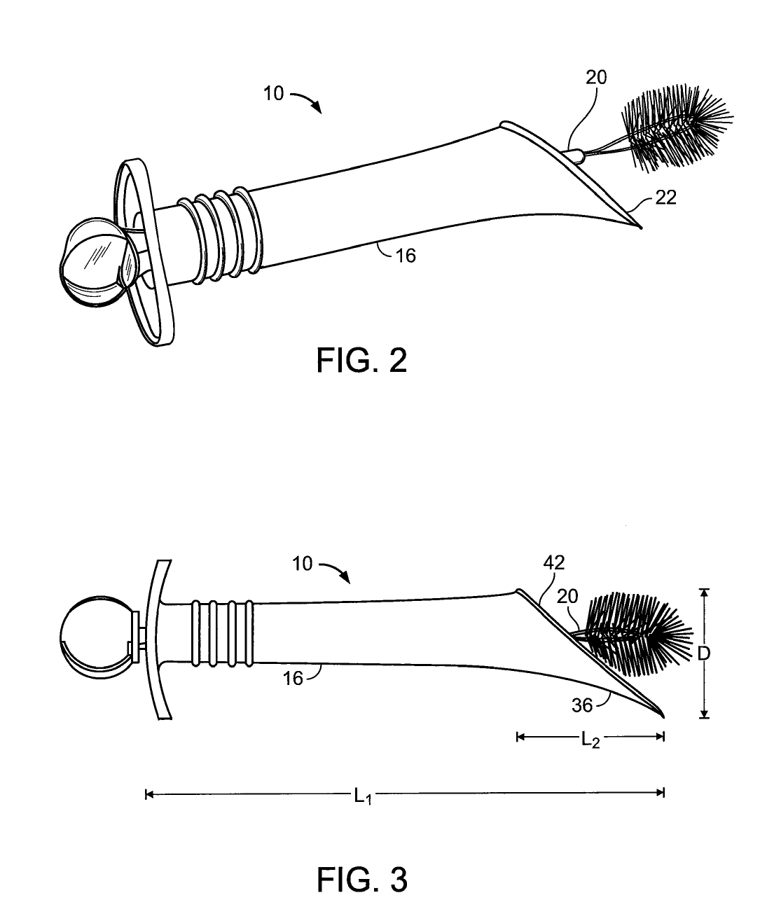

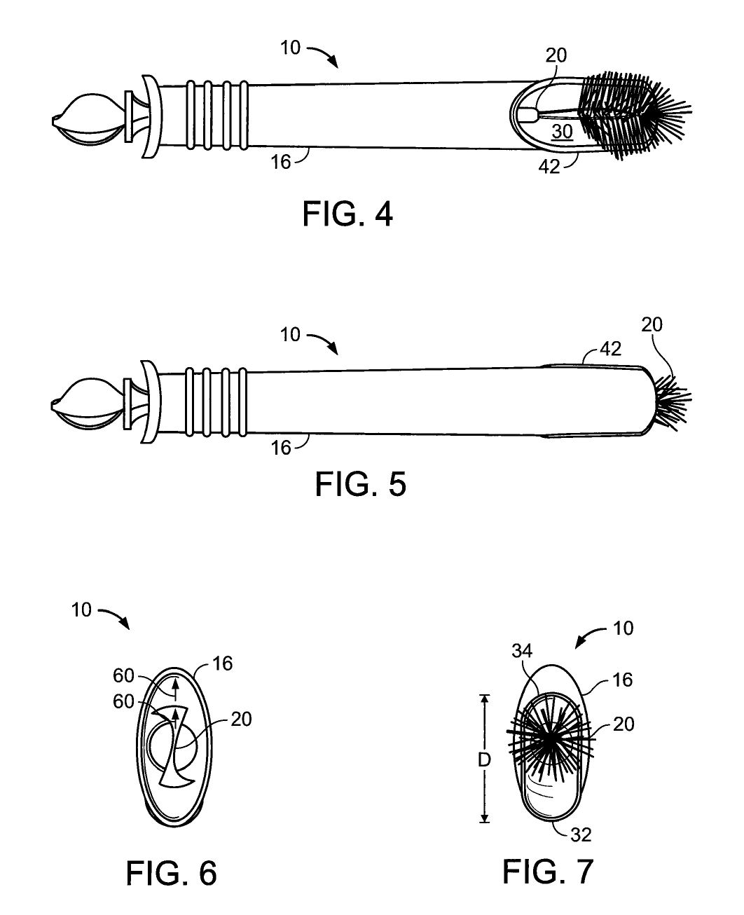

A specimen collection device 10 is illustrated in FIG. 1 accessing the cervix 12 to collect cells and secretions from the area of the cervical os 14. The specimen collection device 10 includes a hollow, tubular member 16 of fixed dimension sized for placement in the vaginal canal 18 and a sampling member 20 configured for receipt within the tubular member 16. The tubular member has a length, l₁, e.g., in the range of 120 to 220 mm, sufficient to extend from the cervix 12 out of the vagina canal 18. Referring also to FIGS. 2-7, the tubular member 16 flares distally to a fixed, enlarged outer diameter, D, in the range of 30 to 50 mm, that acts to retain the tubular member 16 in a desired location within the vaginal canal during use. Sampling member 20 extends out of an obliquely angled, distal end 22 of the tubular member 16 into contact with cervix to remove and retain specimen. Device 10 is advantageously configured such that the women from whom the specimen is being collected can perform the collection herself in a home setting.

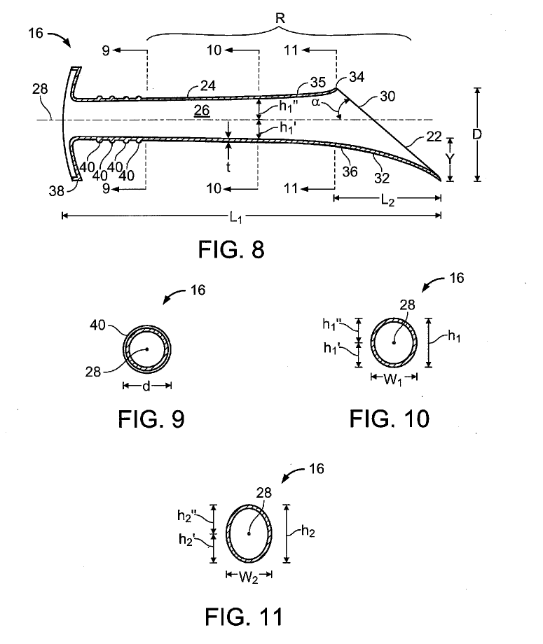

Referring to FIG. 8, the tubular member 16 has a wall 24 defining a longitudinal through channel 26 having a central axis 28. The tubular member 16 defines an opening 30 at the distal end 22 oriented at an oblique angle, α, e.g., 20-65°, to the central axis 28 at the distal end 22 such that a first section 32 of the wall 22 extends distally of a distal end 34 of a second section 35 of the wall 24 by a length, l₂, e.g., 20-60 mm, and terminates at the opening 30. At least a portion of the first section 32 flares distally to an enlarged outer diameter to form a flare 36. As illustrated, no portion of the first section 32 converges toward the second section 34, but rather diverges by an amount, Y, in the range of 13-30 mm to form the flare 36, with the outer surface of the wall 24 being continuous to the distal opening 30. The tubular member 16 also includes a handle 38 to aid in insertion of the tubular member 16 into the vaginal canal.

Along the length of the tubular member 16 is a region, R, with an asymmetric, longitudinal cross-section formed by the wall 24 flaring in opposite directions at different rates. Referring to FIGS. 9-11, in an exemplary configuration, the cross-section of the tubular member 16 changes from circular (FIG. 9) with a diameter, d, of 9-12 mm, to asymmetric (FIGS. 10 and 11). The central axis 28 is defined by the central axis of the tubular member in the region having a circular cross-section, and, in the illustrated embodiment, extends the length of the tubular member without curving. As seen in FIGS. 10 and 11, the cross-sectional shape becomes increasingly oblong with a height, h₁, of 10-14 mm, and a width, w₁, of 9-13 mm at the cross-section of FIG. 10, and a height, h₂, of 12-17 mm, and a width, w₂, of 9-15 mm at the cross-section of FIG. 11. At the cross-sections of FIGS. 10 and 11, the height dimension is asymmetric about the central axis 28 (for example, h₁′ and h₂′ are 1-3 mm longer that h₁″ and h₂″, respectively, with the difference being more pronounced at the cross-section of FIG. 11.), while the width dimension is symmetric about the central axis 28.

The ratio of diameters D/d is, for example, 5 times or less. The overall change in the outer diameter of the tubular member 16 over the length of the flaring is, for example, from a smaller diameter in the range of 15-20 mm to a larger diameter in the range of 30-50 mm. The wall thickness, t, for example, 2-5 mm, of the tubular member 16, remains constant along the length of the tubular member except for the addition of circumferential handling nubs 40, and a thickened bead 42 (FIGS. 3-5) around the distal end 22 of the tubular member 16 defining the opening 30. The thickened bead 42 provides a smooth, rounded edge on the tubular member 16 to limit any possibility of damaging tissue during advancement of the tubular member into the vaginal canal and while the tubular member is residing in the vaginal canal.

The diameter, D, at the end of the flare 36 is a projected, effective outer diameter measured from the ends of the first and second circumferential sections 32, 34. The flare to this diameter provides the desired amount of stretching of the vaginal wall to hold the tubular member 16 in place such that the tubular member 16 establishes a relatively fixed and optimal frame of reference to ensure the subsequent/coincident positional accuracy of the specimen removing and retaining element 54 of the sampling member 20 with respect to the targeted cervicovaginal tissue of interest. In addition, due to the obliquely angled distal end 22 of the tubular member 16, the user is not subjected to inserting what would amount to a larger device having an actual, non-projected outer diameter, D.

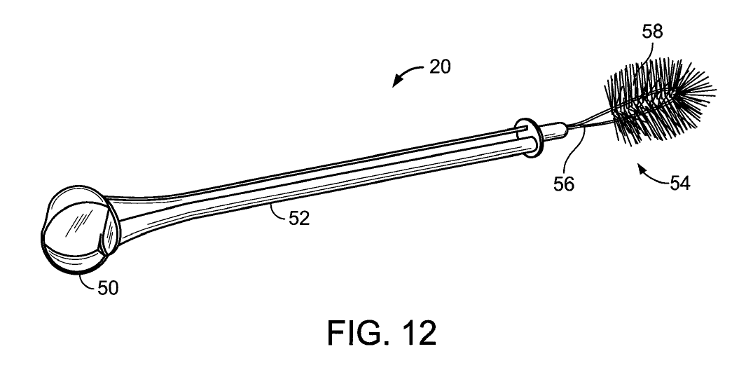

Referring to FIG. 12, the sampling member 20 includes a handle 50, a shaft 52, and a specimen removing and retaining element in the form of a brush 54 oriented off-axis relative to the shaft 52. The brush 54 includes a wire loop 56 to which are attached bristles 58. Brush 54 acts to exfoliate and absorb cells and secretions. The off-axis orientation of the brush 54 biases the brush toward the cervix with the outer tubular member 16 orientated as shown in FIG. 1. The sampling member 20 has a length of about 170-270 mm, for example, about 50 mm longer than the tubular member 16, which is sufficient to extend from the cervix out of the vagina, and is actuatable by the user from outside the vaginal canal to contact the cervix to collect specimen. One or both of the outer tubular member 16 and the sampling member 20 can include orientation markers 60 (FIG. 6) to help position members 16 and 20 relative to each other and to align device 10 with the vaginal canal.

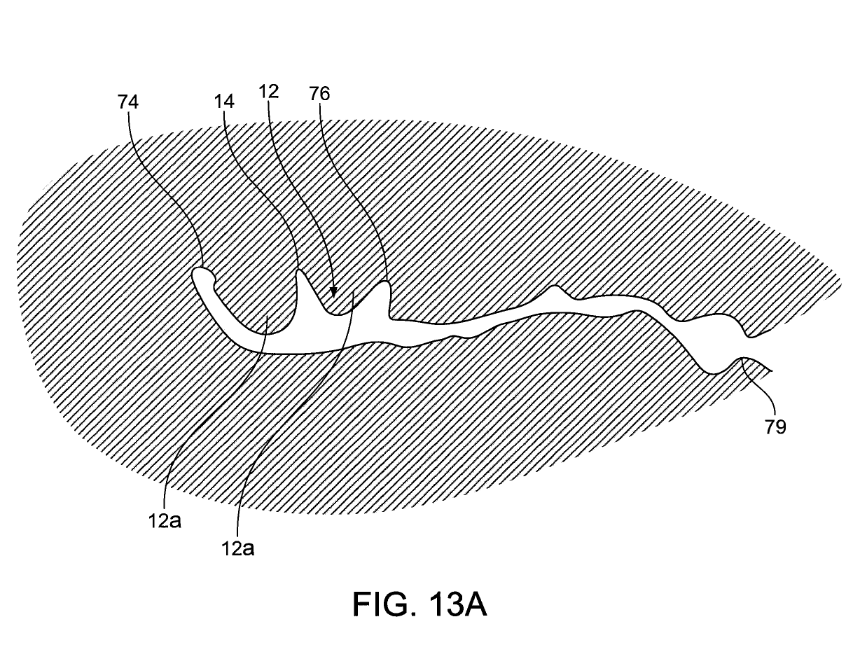

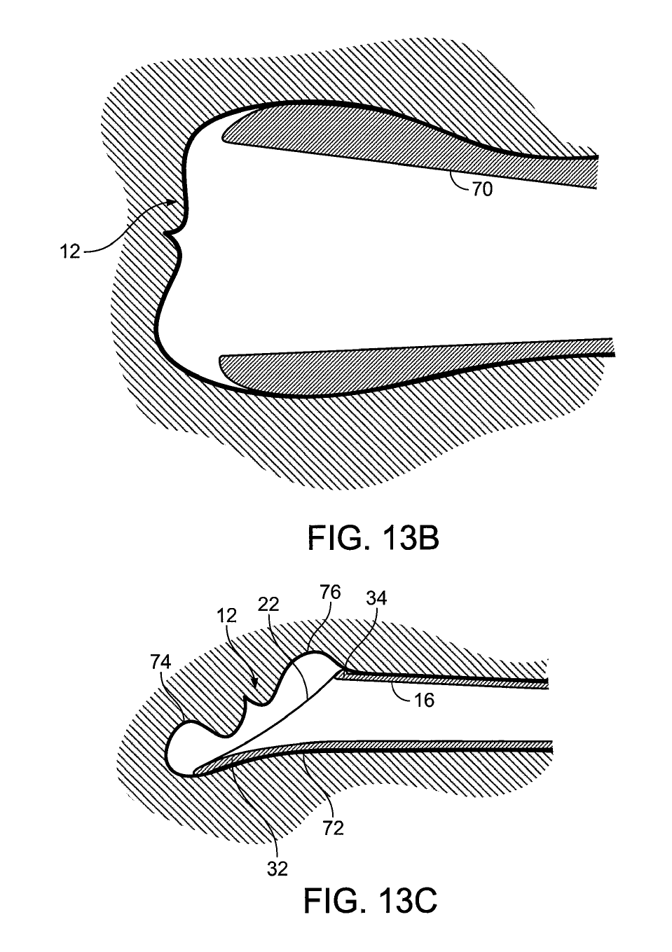

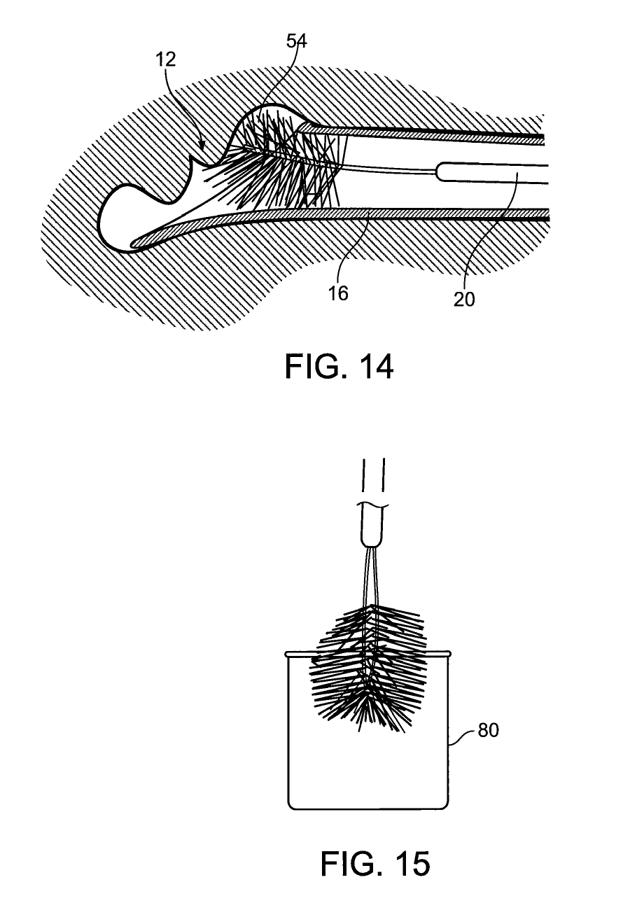

The anatomy of the human vaginal canal and cervix is illustrated in FIG. 13A. The portion of the cervix 12 that projects in the vaginal canal is referred to as the portio vaginalis 12a. FIG. 13B shows the vaginal canal widened by a typical speculum 70 used to access the cervix with the expanded speculum providing a “straight shot” to the cervix. The anatomy of the vaginal canal and cervix with the tubular member 16 in place is shown in FIG. 13C. Rather than the cervix being oriented transversely to the vaginal canal, as in FIG. 13B, differential pressure applied by the tubular member in the region of the anterior fornix 76 relative to the posterior fornix 74 causes the cervix to rotate forward from its natural position of FIG. 13A to the position illustrated in FIG. 13C. The obliquely angled distal end 22 of the tubular member 16 aligns the opening 30 of the tubular member with the orientation of the titled cervix. The circumferential region 32 of the tubular member 16 also acts to isolate the cervix from the opposite wall 72 of the vaginal canal. As illustrated in FIG. 14, the off-axis orientation of the brush 54 biases the brush toward the cervix 12.

In use to collect cells and secretion from the cervix, a woman in a home setting accesses the cervix by introducing the tubular member 16 into the vaginal canal through the introitus 79 (FIG. 13A) without folding the tubular member, and advancing the tubular member 16 until further advancement is limited by the anatomy of the anterior fornix 76 (FIG. 13A). The tubular member 16 causes the cervix to rotate, reorienting the cervix as shown in FIG. 13C such that the opening 30 faces the cervix, with the tubular member wall 24 isolating the cervix, and stretches the vaginal canal in the vicinity of the cervix to hold the tubular member 16 in position to establish a relatively fixed and optimal frame of reference to ensure the subsequent/coincident positional accuracy of the specimen removing and retaining element 54 of the sampling member 20 with respect to the targeted cervicovaginal tissue of interest. The woman advances the specimen collector 20 through the tubular member into contact with the cervix and moves the specimen collector repetitively back and forth in an axial motion against the cervix to collect the specimen. After the specimen is collected, the woman removes the device 10 from the vaginal canal, removes the sampling member 20 from the tubular member 16, and dips the brush 54 into a collection medium (FIG. 15) and shakes or agitates the brush 54. The collection medium is selected based on the desired sample testing to be performed. The user then sends the vial 80 containing the sample in the collection medium to a testing laboratory.

The sampling member 20 can either be positioned within the tubular member 16 during introduction of the tubular member 16 into the vaginal canal, with the brush 54 located within the tubular member, or the sampling member 20 can be advanced into and through the tubular member 16 after placement of the tubular member 16 in the vaginal canal.

Tubular member 16 is preferably made from commodity, medical-grade thermoplastics, evidencing the following desired/relevant characteristics: low cost; lightweight; strong; excellent dimensional stability; low coefficient of friction/inherent lubricity; soft touch; and dye-dopable. Examples include: High-density Polyethylene (HDPE); High-density Polypropylene (HDPP); Polytetrafluoroethylene (PTFE— i.e., Teflon®); and Rigid Polyvinyl Chloride (PVC). The tubular member is non-foldable in use, as discussed above, by which is meant that in normal use under forces applied by hand it will not collapse, flatten, or fold in on itself, but flexibility and cushioning can be provided.

Brush bristles 58 are preferably made from common, man-made fibers/filaments, evidencing the following desired/relevant characteristics: low cost, good balance between flexibility (for form-fitting in and around unique contours) and rigidity (for ultimate surface friction), ability to create micro-abrasive/scaly surface texture (either mechanically or chemically) for more effective specimen dislodging/exfoliation/removal and trapping/retention. To achieve closer to the ideal effect, the following bristle variables/parameters can be adjusted: round vs. rectangular vs. diamond-shaped vs. hexagonal bristle cross-section; rounded vs. blunt vs. pointed bristle tip; embossed vs. feathered vs. coated bristle shaft; wavy (for bushier brush head) vs. straight bristle shaft. Material examples include: Nylon, Polyethylene, Polypropylene, and DuPont's Herox® and Tynex®.

The brush wire 56 is preferably made from medical/surgical-grade stainless steel; specifically, chosen from the “austenitic” steel family, for example, Type 302 or 304 stainless steel. The brush wire is, for example, in a twisted-wire, closed loop configuration, so as not to expose any rough-cut ends, or even a “blunt point”, which could cause sensitive tissue trauma, and attendant pain, bruising, bleeding, and/or infection. The closed loop presents a rounded/benign tip which simply (and “atraumatically”) crumples/collapses into itself, if inadvertently pushed too hard against the anatomy. Optimization variables include: wire gauge and chemical treatments for strength, flexibility, and durability; single stem/single spiral vs. double stem/single spiral vs. double stem/double spiral configurations to control bushiness and flexibility; and spirals per inch also to control bushiness.

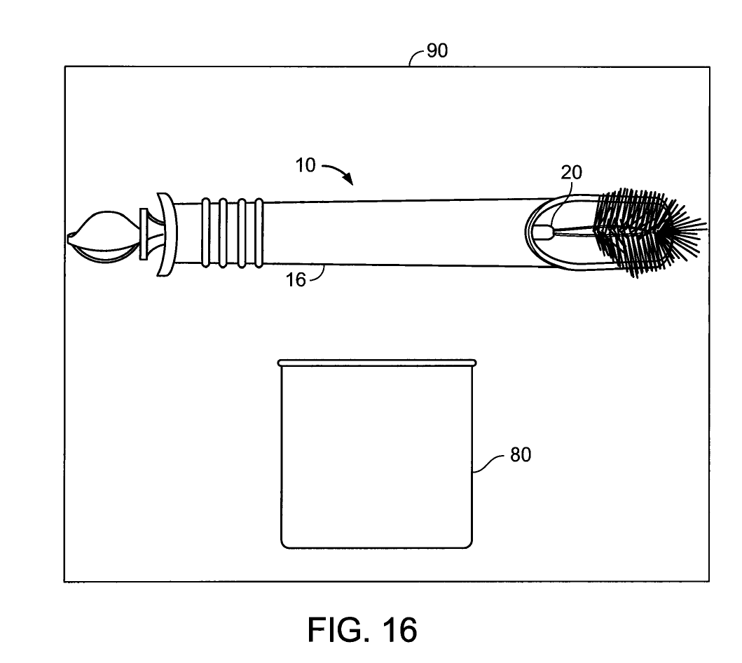

As illustrated in FIG. 16, the specimen collection device 10 and the vial 80 containing the collection medium can be provided as a kit 90. The collection medium is suitable for preserving and transporting human tissue cells and related tissue secretions, for example, PreservCyt® from Cytyc Corporation (Foxboro, Mass.), SurePath® Perservative Solution from TriPath Imaging Inc. (Burlington, N.C.), and CytoRich® from Thermo Scientific division of Thermo Fisher Scientific, Inc. (Waltham, Mass.). To test for high-risk human papillomavirus (HPV), chlamydia (CT) and gonorrhea (GC), the collection medium can be the Rapid Capture® System specimen transport medium (STM) from Digene Corporation (Gaithersburg, Md.). The specimen collection device 10 can be provided in the kit 90 with the tubular member 16 and the sampling member 20 assembled, as illustrated, or separated.

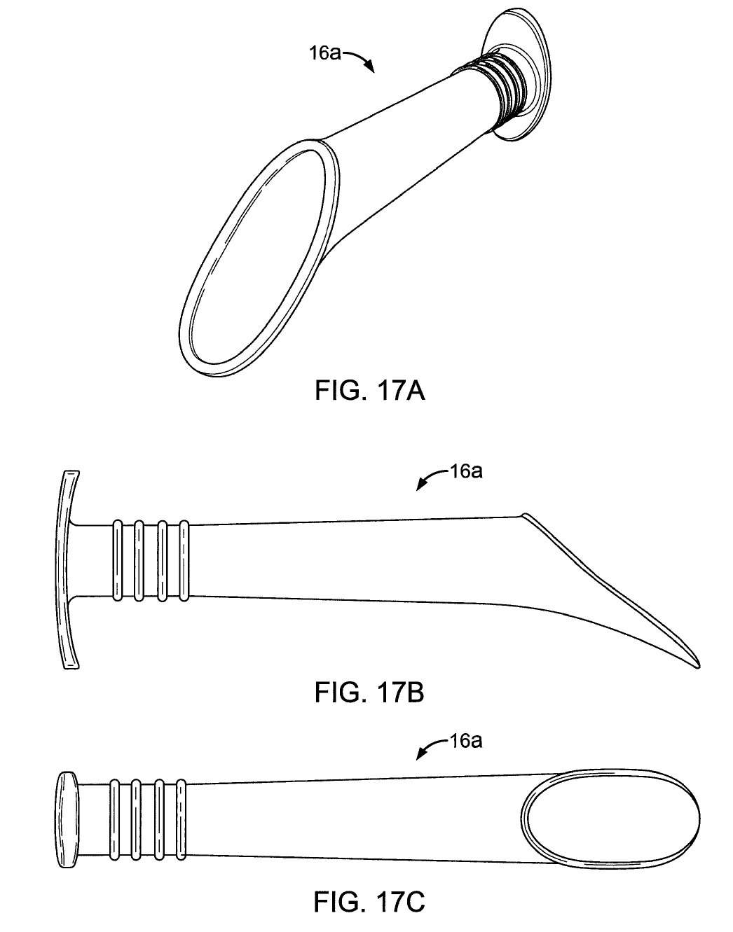

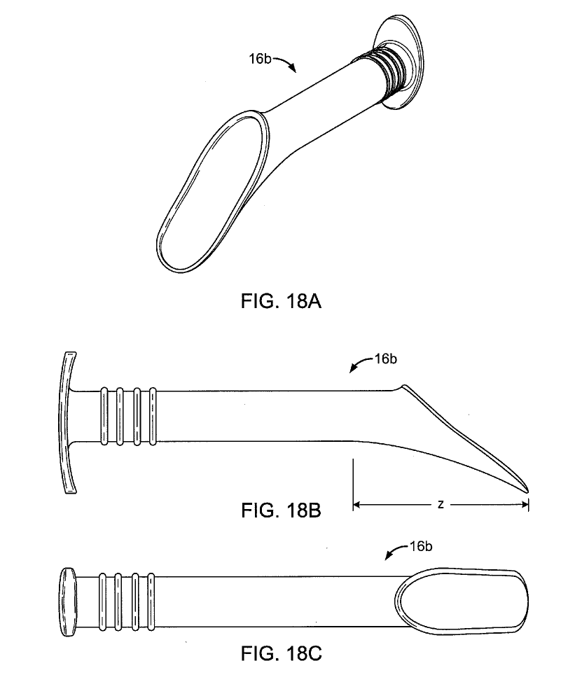

A number of embodiments of the invention have been described. Nevertheless, it will be understood that various modifications may be made without departing from the spirit and scope of the invention. For example, the tubular member 16 need not flare asymmetrically, that is h1′ and h2′ can equal h1″ and h2″, respectively, or the tubular member 16a (FIGS. 17a-17c) can flare conically. The outer diameter of the tubular member can be any of the above described shaped while the inner diameter remain circular. The wall thickness of the tubular member 16 need not be constant. The flaring of the tubular member 16 could begin at a more distal or more proximal location, for example, the wall of the tubular member 16b (FIGS. 18a-18b) can flare just in the distal region, Z, of the tubular member, or just where the first section 32 (FIG. 8) of the wall 22 extends distally of the second section 34, with the remainder of the outer diameter of the tubular member remaining, for example, circular or oblong. The central axis of the tubular member can curve. The tubular member can be used like a lever to apply further downward pressure on the distal end of the tubular member to provide more space for the brush under the cervix. The tubular member can be used for purposes other than specimen collecting. Accordingly, other embodiments are within the scope of the following claims.

1. A device, comprising:
a tubular member sized for placement in the vaginal canal, the tubular member having a wall defining a longitudinal through-channel having a central axis and a line of sight therethrough to a distal end,
the tubular member defining an opening at the distal end oriented at an oblique angle to the central axis such that a first section of the wall extends distally of a distal end of a second section of the wall, the first section flaring outwardly by 13-30 mm to an enlarged outer diameter at the openings,
wherein a circumference of the tubular member in at least a region of the tubular member flaring in opposite directions is asymmetric about the central axis, the asymmetric region having a change in shape along a length of the tubular member.

2. The device of claim 1 wherein the oblique angle comprises 20-65°.

3. The device of claim 1 wherein the first section of the wall extends distally of the distal end of the second section of the wall by 20-60 mm.

4. (canceled)

5. (canceled)

6. The device of claim 1 wherein the opening is bounded by a rounded edge.

7. (canceled)

8. (canceled)

9. The device of claim 1 wherein the tubular member has a length in the range of 120 to 220 mm.

10. The device of claim 1 wherein the tubular member has an outer diameter that flares from a smaller diameter in the range of 15-20 mm to a larger diameter, D, in the range of 30-50 mm.

11. The device of claim 1 further comprising a sampling member configured for receipt within the tubular member.

12. The device of claim 11 wherein the sampling member has a distal end comprising a specimen removing and retaining element.

13. The device of claim 12 wherein the specimen removing and retaining clement comprises a brush.

14. The device of claim 13 wherein the brush is oriented off-axis relative to a longitudinal axis of the sampling member.

15. The device of claim 11 further comprising an orientation indicator on one or both of the tubular member and the sampling member.

16. The device of claim 1 wherein the wall has an outer surface that is continuous to the distal opening.

17. The device of claim 1 wherein the enlarged outer diameter at the opening is a fixed, enlarged outer diameter.

18. A device, comprising:
a tubular member sized for placement in the vaginal canal,
the tubular member having a wall defining a longitudinal through-channel having a central axis,
the tubular member defining an opening at a distal end oriented at an oblique angle to the central axis such that a first section of the wall extends distally of a distal end of a second section of the wall, the first section flaring outwardly by 13-30 mm to an enlarged outer diameter at the opening to form an outer, concave surface of the wall,
wherein a circumference of the tubular member in at least a region of the tubular member flaring in opposite directions is asymmetric about the central axis, the asymmetric region having a change in shape along a length of the tubular member.

19. The device of claim 18 wherein the oblique angle comprises 20-65°.

20. The device of claim 18 wherein the first section of the wall extends distally of the distal end of the second section of the wall by 20-60 mm.

21. The device of claim 18 wherein the opening is bounded by a rounded edge.

22. (canceled)

23. (canceled)

24. The device of claim 18 wherein the tubular member has a length in the range of 120 to 220 mm.

25. The device of claim 18 wherein the tubular member has an outer diameter that flares from a smaller diameter in the range of 15-20 mm to a larger diameter, D, in the range of 30-50 mm.

26. The device of claim 18 further comprising a sampling member configured for receipt within the tubular member.

27. The device of claim 26 wherein the sampling member has a distal end comprising a specimen removing and retaining element.

28. The device of claim 27 wherein the specimen removing and retaining element comprises a brush.

29. The device of claim 28 wherein the brush is oriented off-axis relative to a longitudinal axis of the sampling member.

30. The device of claim 26 further comprising an orientation indicator on one or both of the tubular member and the sampling member.

31. The device of claim 18 wherein the wall has an outer surface that is continuous to the distal opening.

32. The device of claim 18 wherein the enlarged outer diameter at the opening is a fixed, enlarged outer diameter.

33. A device, comprising:
a tubular member sized for placement in the vaginal canal,
the tubular member having a wall defining a longitudinal through-channel having a central axis and a line of sight therethrough to a distal end,
the tubular member defining an opening at the distal end oriented at an oblique angle to the central axis such that a first section of the wall extends distally of a distal end of a second section of the wall, the first section flaring outwardly by 13-30 mm to an enlarged outer diameter at the opening,
the tubular member having an outer diameter that flares from a smaller diameter in the range of 15-20 mm to a larger diameter, D, in the range of 30-50 mm.

34. The device of claim 33 wherein the oblique angle comprises 20-65°.

35. The device of claim 33 wherein the first section of the wall extends distally of the distal end of the second section of the wall by 20-60 mm.

36. The device of claim 33 wherein the opening is bounded by a rounded edge.

37. The device of claim 33 wherein a circumference of the tubular member in at least a region of the tubular member flaring in opposite directions is asymmetric about the central axis.

38. The device of claim 33 wherein the tubular member has a length in the range of 120 to 220 mm.

39. The device of claim 33 further comprising a sampling member configured for receipt within the tubular member.

40. The device of claim 39 wherein the sampling member has a distal end comprising a specimen removing and retaining element.

41. The device of claim 40 wherein the specimen removing and retaining element comprises a brush.

42. The device of claim 41 wherein the brush is oriented off-axis relative to a longitudinal axis of the sampling member.

43. The device of claim 39 further comprising an orientation indicator on one or both of the tubular member and the sampling member.

44. The device of claim 33 wherein the wall has an outer surface that is continuous to the distal opening.

45. The device of claim 33 wherein the enlarged outer diameter at the opening is a fixed, enlarged outer diameter.

46. A device, comprising:
a tubular member sized for placement in the vaginal canal,
the tubular member having a wall defining a longitudinal through-channel having a central axis,
the tubular member defining an opening at a distal end oriented at an oblique angle to the central axis such that a first section of the wall extends distally of a distal end of a second section of the wall, the first section flaring outwardly by 13-30 mm to an enlarged outer diameter at the opening to form an outer, concave surface of the wall,
the tubular member having an outer diameter that flares from a smaller diameter in the range of 15-20 mm to a larger diameter, D, in the range of 30-50 mm.

47. The device of claim 46 wherein the oblique angle comprises 20-65°.

48. The device of claim 46 wherein the first section of the wall extends distally of the distal end of the second section of the wall by 20-60 mm.

49. The device of claim 46 wherein the opening is bounded by a rounded edge.

50. The device of claim 46 wherein a circumference of the tubular member in at least a region of the tubular member flaring in opposite directions is asymmetric about the central axis.

51. The device of claim 46 wherein the tubular member has a length in the range of 120 to 220 mm.

52. The device of claim 46 further comprising a sampling member configured for receipt within the tubular member.

53. The device of claim 52 wherein the sampling member has a distal end comprising a specimen removing and retaining element.

54. The device of claim 53 wherein the specimen removing and retaining element comprises a brush.

55. The device of claim 54 wherein the brush is oriented off-axis relative to a longitudinal axis of the sampling member.

56. The device of claim 52 further comprising an orientation indicator on one or both of the tubular member and the sampling member.

57. The device of claim 46 wherein the wall has an outer surface that is continuous to the distal opening.

58. The device of claim 46 wherein the enlarged outer diameter at the opening is a fixed, enlarged outer diameter.
